(19) United States
(12) Patent Application Publication
(10) Pub. No.: US 2004/0092985 A1
(43) Pub. Date: May 13, 2004

(54) Device for providing thoracoscopic intracardiac access

(75) Inventors: Shailendra K. Parihar, Coopersburg, PA (US); Rajesh Pendekanti, Bridgewater, NJ (US); David Stoloff, Whitehouse Station, NJ (US)

Correspondence Address: SCULLY SCOTT MURPHY & PRESSER, PC, 400 GARDEN CITY PLAZA, GARDEN CITY, NY, 11530

(73) Assignee: ETHICON, INC., Somerville, NJ

(21) Appl. No.: 10/261,430

(22) Filed: Sep. 30, 2002

(51) Int. Cl. A61B 17/32

(52) U.S. Cl. 606/167

(57) ABSTRACT

Devices and methods are provided for providing endoscopic access into an interior of a hollow organ. The method and devices secure a distal end of an access device to seal and secure the distal end to a wall of the hollow organ and secure a proximal end to a skin of the patient. Alternatively, the distal end of the access device is sealed to the wall of the heart and a column of blood from the heart is maintained in at least a portion of the access device to reduce the possibility of introducing an embolism into the heart. In another alternative, the distal end of the access device is disposed on a wall of the hollow organ and vacuum-sealed thereto with a suction cup disposed at the distal end of the access device to secure and seal the distal end to the wall of the hollow organ.

### BACKGROUND OF THE INVENTION

1. Field of the Invention

The present invention relates generally to medical devices, and more particularly, to a multifunctional access device for providing a sealed access to an interior of a hollow organ.

2. Prior Art

Ordinarily surgery to correct heart defects such as atrial septal defect (ASD), ventricular septal defect (VSD), patent ductus arteriosis (PDA), and Atrial Fibrillation (AF) are performed using open-chest techniques while the heart is under cardioplegic arrest and circulation is maintained by cardiopulmonary bypass. Using such techniques, a gross thoracotomy is created in order to gain access to the heart and great vessels, facilitating clamping and cannulation of the aorta for inducing cardioplegic arrest, and allowing instruments to be introduced into the chest cavity and into the heart to perform the surgical repair. The necessity of stopping the heart significantly heightens the risks attendant such procedures, particularly the risks of causing ischemic damage to the heart muscle, and of causing stroke or other injury due to circulatory emboli produced by aortic clamping and vascular cannulation. In addition, the creation of a gross thoracotomy produces significant morbidity and mortality, lengthens hospital stays and subsequent recovery times, increases costs, and results in an increase in the pain and trauma suffered by the patient.

A number of endovascular approaches have been developed which attempt to allow intracardiac access using catheters introduced transluminally from peripheral vessels into the heart. However, these endovascular devices suffer from many problems, including a lack of control and precise positionability from the proximal end of the highly flexible and elongated devices, the significant size constraints of peripheral vessels, and the inability to position the devices in all potentially diseased sites within the heart.

A number of minimally invasive or endoscopic access devices for use in beating heart procedures have also been developed in the prior art. These endoscopic devices are used to gain intracardiac access to the heart. Such devices are disclosed in U.S. Pat. Nos. 6,079,414 to Roth and U.S. Pat. No. 5,829,447 to Stevens et al., which are incorporated herein by their reference. Such devices generally have a substantially long axial bore into which instruments are passed. However, such endoscopic access devices cannot be secured to the heart wall to maintain a tight seal with the heart wall and do not provide any means for controlling circulatory emboli caused by air leakage into the heart.

### SUMMARY OF THE INVENTION

Therefore it is an object of the present invention to provide an access device for providing access to an interior of a hollow organ that overcomes the disadvantages of the prior art.

Accordingly, an access device for providing endoscopic access into an interior of a hollow organ of a patient is provided. The access device comprising: a first tubular body, the first tubular body extending from a proximal end at an exterior surface of the patient to a distal end at the hollow organ, the first tubular body further having a first conduit for passage of an instrument from the proximal to the distal end; distal end securing means for at least one of sealing and securing the distal end of the first tubular body to a wall of the hollow organ; and proximal end securing means for securing the proximal end of the first tubular body to a skin of the patient.

Preferably, the first tubular body further comprises a proximal seal on the proximal end for sealing against an instrument passed through the conduit. The proximal seal is preferably an elastomer boot having a second conduit in fluid communication with the first conduit and an opening to the second conduit having a size substantially equivalent to an outer size of the instrument.

The access device preferably further comprises at least one access port in communication with the first conduit. Preferably, the at least one access port comprises a y-fitting disposed at the proximal end of the first tubular body, the y-fitting having first and second access ports, each of which having a channel in communication with the first conduit. Preferably, at least one of the first and second access ports further comprises an accessory seal disposed in or on the corresponding channel for sealing against an accessory instrument inserted therein. The accessory seal preferably comprises a sealing cap having a bore with at least a portion of the bore substantially corresponding to an outer size of the accessory instrument, the sealing cap further having a slot extending from an exterior surface of the sealing cap to the bore for facilitating insertion of the accessory instrument into the bore.

Preferably, the distal end securing means at least secures the distal end to the wall of the hollow organ and wherein the distal end securing means comprises a suture anchor disposed at the distal end of the first tubular body for anchoring the distal end to the wall of the hollow organ with at least one suture engaging the wall and the suture anchor. The suture anchor preferably has at least one of slits and holes for engaging with the at least one suture.

The distal end securing means preferably at least seals the distal end to the wall of the hollow organ. Preferably, the distal end securing means comprises: a suction cup disposed at the distal end of the first tubular body for engaging with the wall of the hollow organ; and a conduit in fluid communication with an interior of the suction cup for providing a vacuum in the interior of the suction cup to thereby secure and seal the distal end of the first tubular body to the wall of the hollow organ.

Preferably, the proximal end securing means comprises at least one adhesive strip having a free end and a fixed end fixed to the proximal end of the first tubular body, the at least one adhesive strip having an adhesive on at least one surface thereof for attaching to the skin.

Where the hollow organ is the heart, the access device preferably further comprises embolism control means for reducing a possibility of introducing an embolism into the heart. Preferably, the distal end securing means at least seals the distal end to the wall of the heart and the embolism control means comprises a vacuum port in communication with the first conduit for applying a vacuum to the first conduit to draw and maintain a column of blood from the heart into at least a portion of the first conduit. At least a portion of the first tubular body preferably comprises a transparent material for visualizing a height of the column of blood in which case the access device preferably further comprises a marker disposed on the transparent material for indicating a desired height of the column of blood. Preferably, the embolism control means further comprises an anticoagulation coating disposed on at least an interior surface of the first conduit.

The access device preferably further comprises a second tubular body for protection of the suction cup during placement of the distal end on the wall of the hollow organ, the second tubular body having a second conduit in which the first tubular body is disposed, the second tubular body being movably disposed between a first position in which the suction cup is disposed within the second conduit and a second position in which the suction cup is exposed for engagement with the wall of the hollow organ. The distal end securing means preferably at least secures the distal end to the wall of the hollow organ in which case the distal end securing means comprises a suture anchor disposed on the second tubular body for anchoring the distal end to the wall of the hollow organ with at least one suture engaged with the wall and the suture anchor. Preferably, the suture anchor has at least one of slits and holes for engaging with the at least one suture.

Preferably, the distal end of the first tubular body includes an extension portion that extends into the opening in the wall of the hollow organ. The extension portion preferably comprises one or more barbed edges on an exterior of the extension portion of the first tubular body for engaging with the opening in the wall of the hollow organ. Where the distal end securing means at least seals the distal end to the wall of the hollow organ, the distal end securing means preferably comprises: a suction cup disposed at the distal end of the first tubular body for engaging with the wall of the hollow organ; and a conduit in fluid communication with an interior of the suction cup for providing a vacuum in the interior of the suction cup to thereby secure and seal the distal end of the first tubular body to the wall of the hollow organ; wherein the extension portion is distal to the suction cup.

The access device preferably further comprises a second tubular body movably disposed within the first conduit, the second tubular body having a second conduit for passage of the instrument from the proximal end to the distal end, wherein a distal end of the second tubular body includes an extension portion that extends into the opening in the wall of the hollow organ. Preferably, the extension portion comprises one or more barbed edges on an exterior of the extension portion of the second tubular body for engaging with the opening in the wall of the hollow organ. The access device preferably further comprises extension means for extending the extension portion of the second tubular body into the opening in the wall of the hollow organ. The extension means preferably comprises: a nut rotatably retained on the first tubular body, the nut having an internal thread; and the second tubular body having a threaded portion on an exterior surface thereof threadingly engaged with the internal thread of the nut; wherein rotation of the nut causes a relative movement between the first and second tubular bodies. Where the distal end securing means at least seals the distal end to the wall of the hollow organ, the distal end securing means preferably comprises: a suction cup disposed at the distal end of the second tubular body for engaging with the wall of the hollow organ; and a conduit in fluid communication with an interior of the suction cup for providing a vacuum in the interior of the suction cup to thereby secure and seal the distal end of the second tubular body to the wall of the hollow organ; wherein the extension portion is distal to the suction cup when in an extended position.

Preferably, the instrument is an incisor having a cutting blade and disposed in the first conduit such that the cutting blade extends from the distal end of the first tubular body to contact the wall of the hollow organ and to incise the opening therein. The incisor preferably further has a dilator for dilating the opening after incision.

Also provided is a first alternative access device for providing endoscopic access into an interior of a heart of a patient. The first alternative access device comprising: a first tubular body, the first tubular body extending from a proximal end at an exterior surface of the patient to a distal end at the heart, the first tubular body further having a first conduit for passage of an instrument from the proximal to the distal end; distal end securing means for at least one of sealing and securing the distal end of the first tubular body to a wall of the heart; and embolism control means for reducing a possibility of introducing an embolism into the heart.

Preferably, the distal end securing means at least seals the distal end to the wall of the heart and the embolism control means comprises a vacuum port in communication with the first conduit for applying a vacuum to the first conduit to draw a column of blood from the heart into at least a portion of the first conduit. At least a portion of the first tubular body preferably comprises a transparent material for visualizing a height of the column of blood. The alternative access device preferably further comprises a marker disposed on the transparent material for indicating a desired height of the column of blood. The embolism control means preferably further comprises an anticoagulation coating disposed on at least an interior surface of the first conduit.

Also provided is a second alternative access device for providing endoscopic access into an interior of a hollow organ of a patient. The second alternative access device comprising: a first tubular body, the first tubular body extending from a proximal end at an exterior surface of the patient to a distal end at the hollow organ, the first tubular body further having a first conduit for passage of an instrument from the proximal to the distal end; a suction cup disposed at the distal end of the first tubular body for engaging with the wall of the hollow organ; and a conduit in fluid communication with an interior of the suction cup for providing a vacuum in the interior of the suction cup to thereby secure and seal the distal end of the first tubular body to the wall of the hollow organ.

Still provided is a method for providing endoscopic access into an interior of a hollow organ of a patient. The method comprising: at least one of sealing and securing a distal end of an access device to a wall of the hollow organ; and securing a proximal end of the access device to a skin of the patient.

Still further provided is a first alternative method for providing endoscopic access into an interior of a heart of a patient. The first alternative method comprising: sealing a distal end of an access device to a wall of the heart; and maintaining a column of blood from the heart into at least a portion of an access device to reduce a possibility of introducing an embolism into the heart. Preferably, the maintaining comprises applying a vacuum to an instrument conduit in the access device. In which case the method further comprises controlling an amount of vacuum to vary a height of the column of blood in the instrument conduit.

Yet still further provided is a second alternative method for providing endoscopic access into an interior of a hollow organ of a patient. The second alternative method comprising: disposing a distal end of an access device on a wall of the hollow organ; and vacuum sealing the distal end to the wall with a suction cup disposed at the distal end of the access device to thereby secure and seal the distal end to the wall of the hollow organ.

### BRIEF DESCRIPTION OF THE DRAWINGS

These and other features, aspects, and advantages of the apparatus and methods of the present invention will become better understood with regard to the following description, appended claims, and accompanying drawings where:

FIG. 1 illustrates an isometric view of a first preferred implementation of an access device of the present invention.

FIG. 2 illustrates a sectional view of the access device of FIG. 1 as taken along line 2-2 in FIG. 1.

FIG. 3A illustrates a perspective view of a preferred sealing cap for an accessory device for use with an access device of the present invention.

FIG. 3B illustrates a sectional view of a preferred suction cup for use with the access device of FIG. 1.

FIG. 3C illustrates a perspective view of a preferred suture anchor for use with the access device of FIG. 1.

FIGS. 4A-4C schematically illustrate a sequence of operation for the distal end of the access device of FIG. 1 against a wall of a hollow organ.

FIG. 5 illustrates an isometric view of a first alternative implementation of the access device of FIG. 1 having an incisor inserted therein.

FIG. 6 illustrates a sectional view of the access device and incisor of FIG. 5 as taken along line 6-6 in FIG. 5.

FIG. 7 illustrates a section view of a second alternative implementation of the access device of FIG. 1.

FIG. 8 illustrates a distal end of the access device of FIG. 7.

FIG. 9 illustrates an isometric view of a first alternative distal end sealing and securing means for an access device of the present invention, the perspective of the isometric view being altered at one end for clarity.

FIGS. 10A-10D illustrate a sequence of operation for the distal end of the access device of FIG. 9 against a wall of a hollow organ.

FIG. 11 illustrates an isometric view of a second alternative distal end sealing and securing means for an access device of the present invention, the perspective of the isometric view being altered at one end for clarity.

FIGS. 12A-12D illustrate a sequence of operation for the distal end of the access device of FIG. 9 against a wall of a hollow organ.

FIGS. 13A-13C illustrate a sequence of operation for a third alternative distal end sealing and securing means for an access device of the present invention against a wall of a hollow organ.

FIGS. 14A and 14B illustrate a sequence of operation for a variation of the third alternative distal end sealing and securing means of FIGS. 13A-13C.

FIG. 15A illustrates a side view of a fifth alternative distal end sealing and securing means for an access device of the present invention.

FIG. 15B illustrates an enlarged distal end portion of the distal end sealing means of FIG. 15A.

FIGS. 16A-16F illustrate a sequence of operation for the distal end sealing and securing means of FIG. 15A against a wall of a hollow organ.

### DETAILED DESCRIPTION OF THE PREFERRED EMBODIMENT

Although this invention is applicable to numerous and various types of surgical procedures and hollow organs, it has been found particularly useful in the environment of intracardiac access in a thoracoscopic (closed chest) procedure. Therefore, without limiting the applicability of the invention to intracardiac access in a thoracoscopic procedure, the invention will be described in such environment. In general, the devices of the present invention are used in endoscopic, minimally invasive, and/or less invasive procedures (collectively referred to herein as “endoscopic procedures”), preferably, in applications which require an endoscopic access for a device or instrument with hemostasis and embolism control, such as, for the ablation devices used to treat Atrial Fibrillation (AF) in a beating heart or other procedures within the heart while the heart is beating and the chest is closed. Examples of other such instruments are described in U.S. Pat. No. 6,079,414.

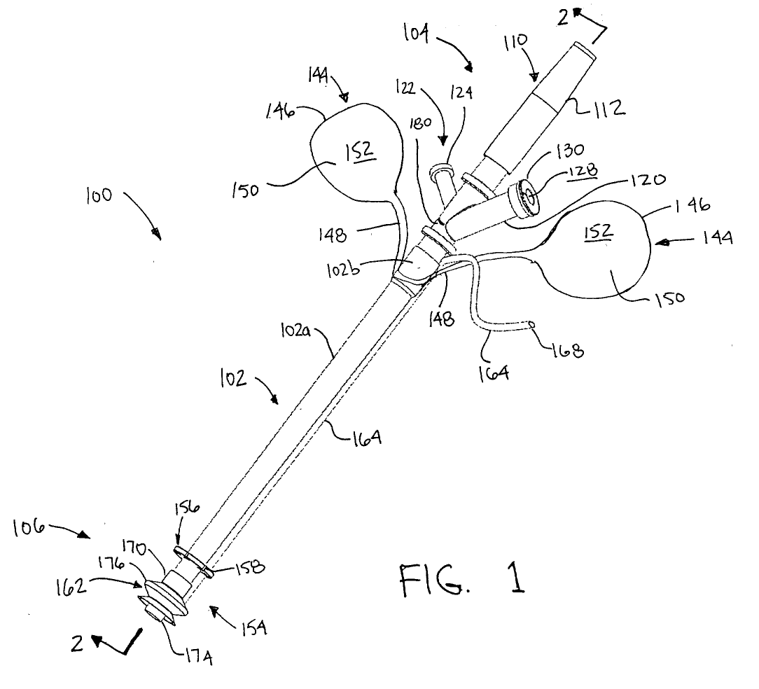

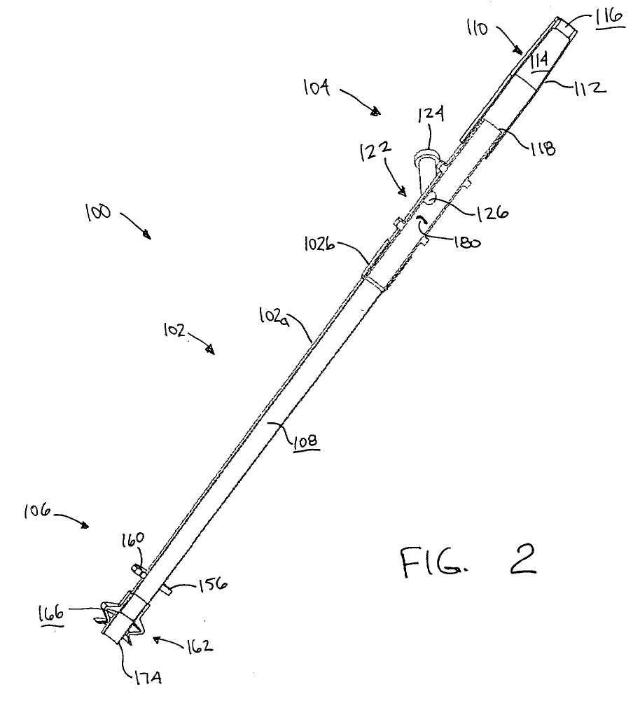

Referring now to FIGS. 1 and 2, there is illustrated a first preferred implementation of an access device of the present invention, generally referred to by reference numeral 100. Access device 100 provides endoscopic access into an interior of a hollow organ, such as the heart, of a patient. The access device 100 includes a first tubular body 102. The first tubular body 102 is sized to extend from a proximal end 104 at an exterior surface of the patient to a distal end 106 at the hollow organ. The first tubular body 102 also has a first conduit 108 for passage of an instrument from the proximal end 104 to the distal end 106. The first tubular body 102 is preferably cylindrical in shape and fabricated from metallic material, such as stainless steel, or a medically approved thermoplastic. The first tubular body 102 can also be fabricated in sections of differing materials, such as a distal portion 102a being fabricated from stainless steel and other portions being fabricated from a thermoplastic and/or elastomers.

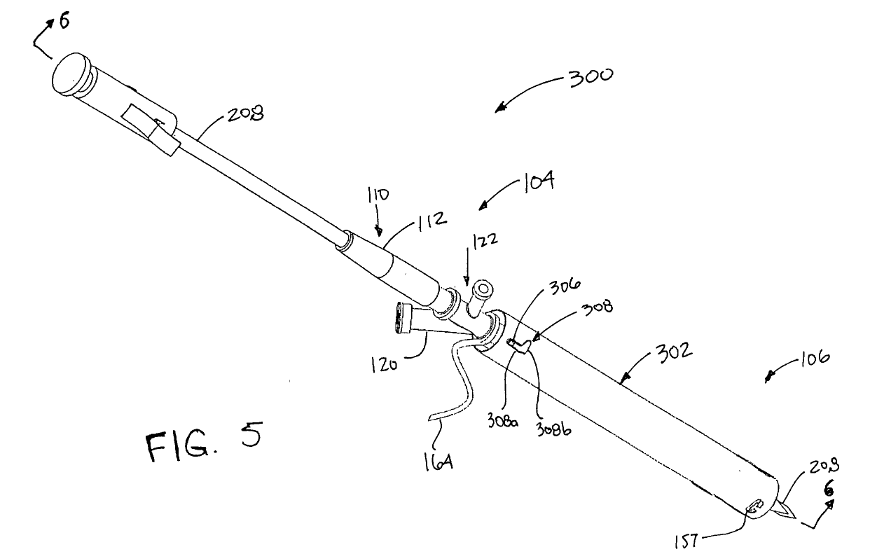

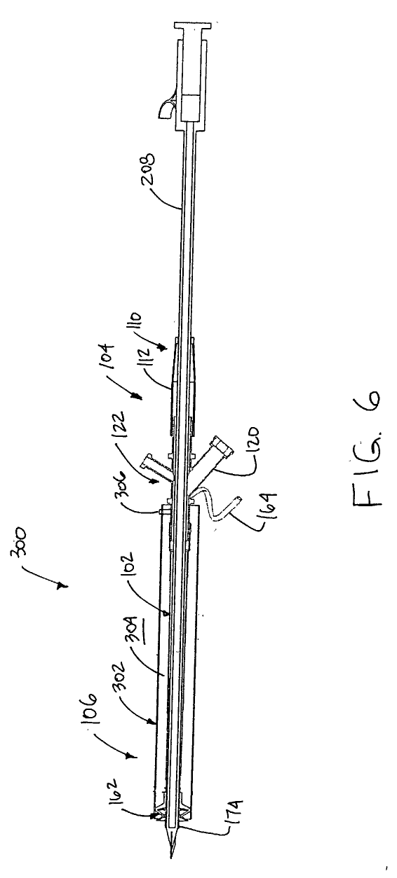

The first tubular body 102 further comprises a proximal seal 110 on the proximal end 104 for sealing against an exterior surface of an instrument (As shown in FIGS. 5 and 6) passed through the first conduit 108. The proximal seal 110 is preferably an elastomer boot 112 having a second conduit 114 in fluid communication with the first conduit 108 and an opening 116 to the second conduit 114 having a size substantially equivalent to an outer size of the instrument. An outer surface of the instrument seals against surfaces defining the opening 116. The elastomer boot 112 is preferably cylindrical as is the opening 116 so as to correspond to a typical cylindrically shaped instrument for use therewith. The elastomer boot 112 is preferably fabricated from any medical grade elastomer, such as silicone and is preferably press-fit over an end 118 of the first tubular body 102. However, the elastomer boot 112 may also be clamped or adhered to the end 118 of the first tubular body 102 or may even be integrally formed therewith. Alternatively, the seal may be internal to the first conduit 108 and may comprise a duckbill valve, as are known in the art. Such an alternative may also be located at the distal end 106 of the access device.

The access device 100 further comprises at least one access port 120 in communication with the first conduit 108 (or alternatively in communication with the second conduit 114 of the elastomer boot 112) for passage of an accessory device, such as an ablation probe (not shown), to the distal end 106 via the first conduit 108. The access port 120 preferably comprises a y-fitting 122 disposed at the proximal end 104 of the first tubular body 102. The y-fitting 122 preferably has a second access port 124, which as will be described below, is useful for applying a vacuum to the first conduit 108. Each of the access ports have an internal channel 126 in communication with the first conduit 108 and an opening 128 in communication with the internal channel 126. The y-fitting 122 is preferably fabricated from a medical grade thermoplastic material, and for reasons described below, is preferably transparent or has a transparent portion. The y-fitting is preferably press-fit into an end 102b of the proximal portion 102a of the first tubular body 102 and/or adhered thereto. The end 102b of the proximal end of the first tubular body 102a is preferably swaged to accommodate the insertion of the y-fitting 122. The y-fitting 122 can alternatively be integrally formed with the first tubular body 102 and/or the elastomer boot 112 such that the first tubular body 102 is essentially a unitary member with the exception of any necessary seals for the instruments and accessories inserted therein, which may be disposed therein or formed therewith.

Where an accessory device is passed through the opening 128 and internal channel 126, it is preferred that the corresponding access port 120 include an accessory seal 130 disposed in or on the corresponding internal channel 126 for sealing against an accessory instrument inserted therein. Preferably, as shown in FIG. 1, the accessory seal 130 is disposed at the opening 128 of the internal channel 126.

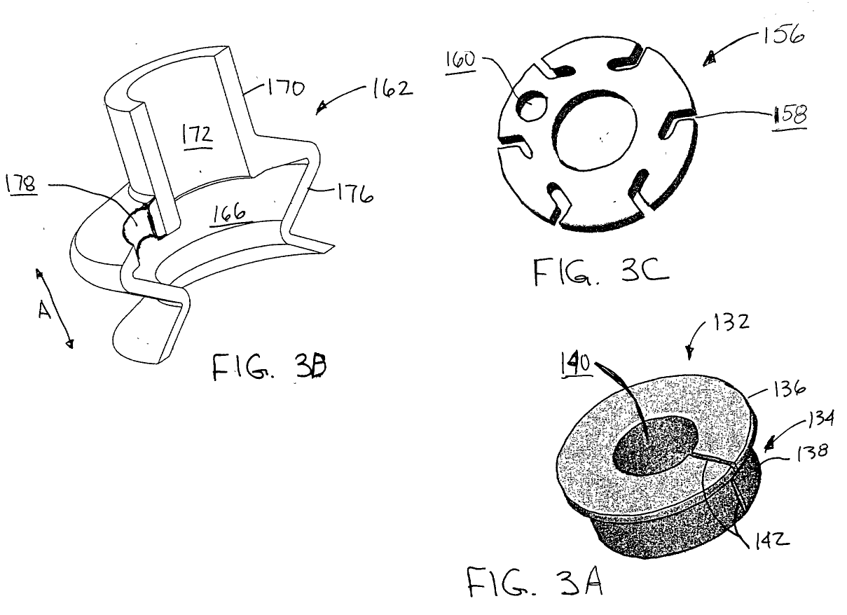

Referring now to FIG. 3A, there is shown a preferred implementation of the accessory seal 130 in the form of a sealing cap 132. The sealing cap 132 has a body 134 having a lipped portion 136 and a press-fit portion 138. The press-fit portion 138 being pressed into the opening 128 of the accessory port 120 with the lipped portion 136 butting against an end of the accessory port 120. The sealing cap 132 also has a bore 140 with at least a portion of the bore 140 substantially corresponding to an outer size of the accessory instrument. The sealing cap 132 further has a slot 142 extending from an exterior surface of the sealing cap 132 to the bore 140 for facilitating insertion of the accessory instrument into the bore 140. In operation, the body 134 is pulled apart to allow the accessory instrument to pass through the slot 142 and into the bore 140. The body 134 is then released and surfaces defining the bore 140 conforms to an outer surface of the accessory instrument, thus, sealing against it. The press-fit portion 138 of the body 134 is then inserted into the opening 128 of the accessory port 120 as described above, which can serve to further urge the surfaces defining the bore 140 against the outer surfaces of the accessory instrument.

Referring back to FIGS. 1 and 2, the access device preferably further includes a proximal end securing means for securing the proximal end 104 of the first tubular body 102 to a skin of the patient. The proximal end securing means preferably comprises at least one flexible adhesive strip 144 having a free end 146 and a fixed end 148 fixed to the proximal end 104 of the first tubular body 102, preferably at the swaged end 102b of the distal portion 102a of the first tubular body 102. Preferably two or more adhesive strips 144 are provided, each having an adhesive 150 on at least one surface 152 thereof for attaching to the skin. The adhesive strips 144 are preferably fabricated from a thin flexible material, such as cloth or plastic film and the adhesive 150 is similar to that used for adhesive bandages known in the art. The surface 152 having the adhesive 150 may also have a peelable layer (not shown), which is removed to expose the adhesive 150 when necessary. Although adhesive strips 144 are preferred, other proximal end securing means may be employed without departing from the scope or spirit of the present invention, such as sutures tied between the skin and a proximal suture anchor (not shown, but similar to the distal suture anchor described immediately below) disposed at the proximal end 104 of the first tubular body 102.

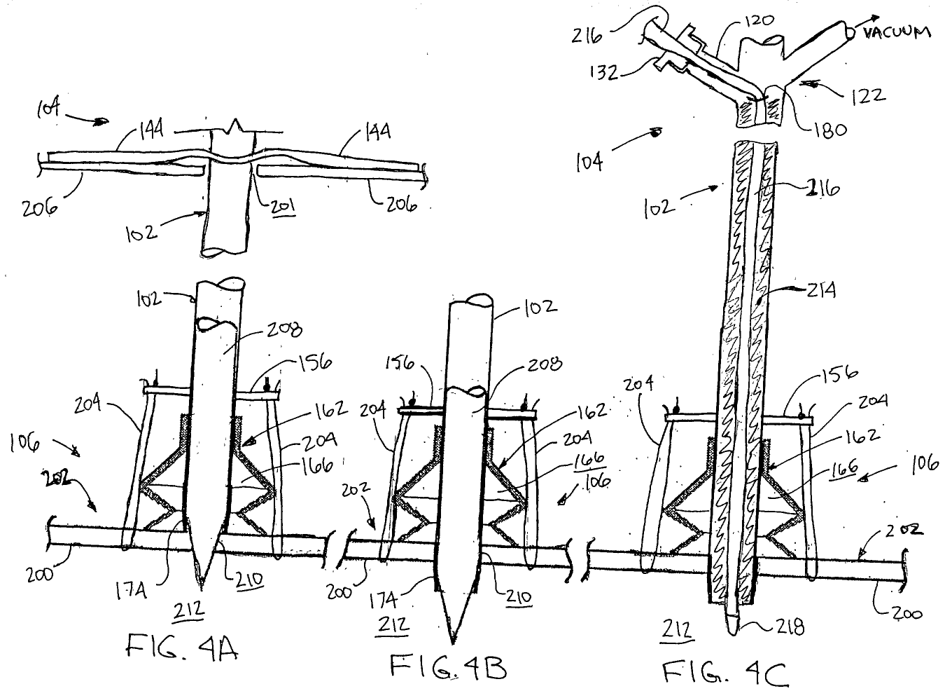

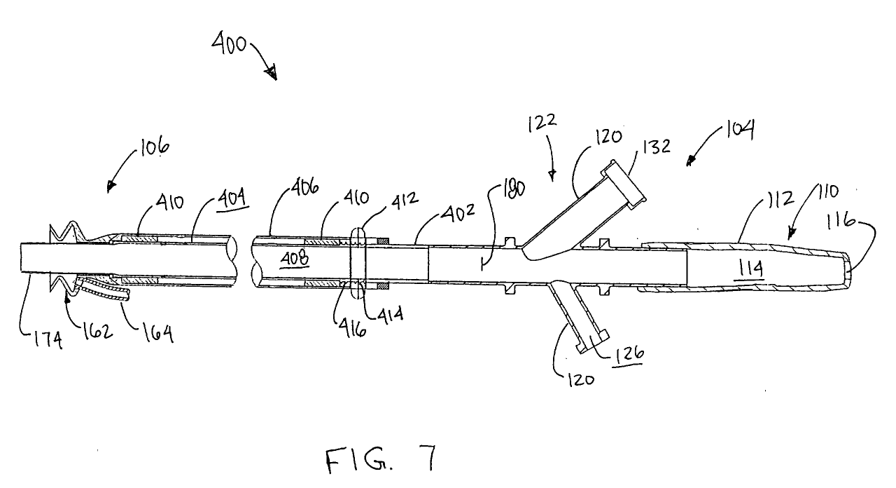

The access device 100 also includes a distal end securing means 154 for at least one of sealing and securing the distal end 106 of the first tubular body 102 to a wall of the hollow organ. Where the distal end securing means 154 only secures the distal end 106 to the wall of the hollow organ, a distal suture anchor 156 is disposed at the distal end 106 of the first tubular body 102 for anchoring the distal end 106 to the wall of the hollow organ with at least one suture (shown in FIGS. 4A-4C) engaging the wall and the suture anchor 156. As shown in FIG. 3C, the suture anchor 156 has at least one of slits 158 and holes 160 for engaging with the at least one suture.

Where the distal end securing means 154 at least seals the distal end 106 to the wall of the hollow organ, a suction cup 162 is disposed at the distal end 106 of the first tubular body 102 for engaging with the wall of the hollow organ. As is shown clearly in FIGS. 7 and 8, a conduit 164, such as a flexible tube, is provided in fluid communication with an interior 166 of the suction cup 162 for providing a vacuum in the interior 166 of the suction cup 162 to thereby secure and seal the distal end 106 of the first tubular body 102 to the wall of the hollow organ. Although, the suction cup 162 both secures and seals the distal end 106 to the wall of the hollow organ, the suture anchor 156 is preferred in combination therewith to provide an initial securement of the distal end 106 before the interior 166 of the suction cup 162 is aspirated. The suture anchor 156 preferably includes a hole for accommodation of the conduit 164. The conduit 164 may be separately fabricated and run alongside the first tubular body 102 or integrally formed with the first tubular body 102 as a double lumen tube (not shown). An end 168 of the conduit 164 is connected to a vacuum source, (not shown) to provide the vacuum in the interior 166 of the suction cup.

Referring now to FIG. 3B, a preferred configuration of the suction cup 162 is illustrated therein. The suction cup 162 is fabricated from a medical grade elastomer, such as silicone and has a bellows shape to facilitate contraction in the A direction. The suction cup 162 has a neck portion 170 having a bore 172 in communication with the interior 166. The distal end 106 of the first tubular body 102 is inserted in the bore 172 and fixed thereto by any means known in the art, such as by an adhesive or a ring clamp (not shown). The suction cup 162 is preferably oriented on the distal end 106 such that an extension portion 174 extends past the suction cup 162. The extension portion 174 preferably has one or more barbed edges 175 (shown in FIG. 8) on an exterior surface for engaging with the opening in the wall of the hollow organ. Upon providing the vacuum to the interior 166 of the suction cup 162 and the resulting contracting thereof, the extension portion 174 is distally thrust further into the opening in the wall of the hollow organ.

The suction cup 162 preferably further has a bellows portion 176 having at least one corrugation to provide the necessary flexibility in the A direction. The bellows portion 176 further has a hole 178 for attachment of the conduit 164, by way of adhesive or any other attachment means known in the art.

Referring back to FIGS. 1 and 2, where the access device 100 is used to access the heart, particularly the left atrium of the heart in a beating heart procedure, it is preferred that the access device 100 include an embolism control means for reducing a possibility of introducing an embolism into the heart. Preferably, the embolism control means comprises sealing the suction cup 162 to the heart wall, sealing any instruments and accessories inserted into the access device 100 as described above, and applying a vacuum to the first conduit 108 through the vacuum port 124 to draw and maintain a column of blood from the heart into at least a portion of the first conduit 108. Preferably, at least a portion of the first tubular body 102, such as the y-fitting 122 comprises a transparent material, such as polycarbonate, for visualizing a height of the column of blood in the first conduit 108. A marker, such as a darkened line 180, is preferably disposed on the transparent material for indicating a desired height of the column of blood. Alternatively, two markers indicating a range for the height of the column of blood can be used. Thus, a vacuum strength can be adjusted until the desired height is achieved. As a further means for embolism control, an anticoagulation coating is disposed on at least an interior surface of the first conduit 108 and preferably on all blood contacting surfaces of the access device 100. The anticoagulation coating can be permanently affixed to the surfaces or applied as a fluid before each procedure.

An operation of the access device 100 will now be described with reference to FIGS. 4A-4C. FIGS. 4A and 4C illustrate a distal end 106 of the access device and a portion of the proximal end 104, while FIG. 4B illustrates only the distal end 106. At the start of a procedure using the access device 100, two or more access incisions are preferably made in the skin, such as on the chest wall to provide access for the access device and a viewing device, such an endoscope (not shown). Although applicable to hollow organs in general, the operation of the access device will be described with regard to the heart, and more preferably, during a beating heart procedure. Preferably, in an intracardiac access procedure, the incision 201 for the access device 100 is made on the back right side of the patient and the access device is advanced to the left atrium of the heart through an intercostal space between the rib cage. A trocar tube (not shown) can be used to protect the suction cup 162 from being damaged during insertion through the ribs. The access device 100 is preferably manipulated under vision provided by the endoscope. Although it is preferred that the endoscope be inserted in a separate incision, it can also be inserted in the first conduit 108.

Before placement of the access device 100 into the incision, appropriate surfaces are coated with an anticoagulation fluid. Before placement of the distal end 108 of the access device 100 on the wall 200 of the heart 202, purse string sutures 204 are endoscopically made, as is known in the art, in the vicinity of the heart wall 200 that is to be incised. The distal end 106 is then placed at the heart wall 202 in the vicinity to be incised and the adhesive strips 144 are adhered to the skin 206 of the patient to secure the proximal end 104 thereto. Once adhered, the y-fitting 122 and elastic boot 112 remain on an exterior of the patient. The purse string sutures 204 are then tied to holes 160 and/or slots 158 of the suture anchor 156 to secure the distal end 106 to the vicinity of the heart wall 200 to be incised. The purse string sutures 204 are preferably tied with another instrument, such as a grasper, located in another port on the skin. Vacuum is then applied to the conduit 164 and interior 166 of the suction cup 162 to seal the distal end 106 to the heart wall 200 and to urge the extension portion 174 against the heart wall 200. It is important for embolism control that no air remains inside the interior 166 of the suction cup 162. A cutting instrument 208 is then inserted into the opening 116 of the elastic boot 112 and slid towards the distal end 106. An accessory instrument (not shown) is inserted into the sealing cap 132 as described above and the sealing cap 132 and accessory instrument are inserted into the opening 128 of the channel 126 of the accessory port 120 such that a distal or working end of the accessory instrument is disposed in the channel 126. Either or both of the cutting instrument and accessory instrument may be pre-loaded in the access device 100 prior to insertion into the incision 201.

Air is then evacuated from interior portions of the access device 100, e.g., the first conduit 108, second conduit 114, and channels 126 by providing a vacuum at one of the accessory ports 120 not being used for an accessory instrument. Referring now to FIG. 4A, the incisor 208 is used to make an incision 210 in the heart wall 200. The incisor may have a sharp leading edge for such purpose or a retractable knife blade (not shown). The incisor 208 is also preferably used to dilate the incision 210 as shown in FIG. 4B such that the extension portion 174 protrudes into the incision 210 and partially into an interior 212 of the heart 202 with the barbed edges 175 (FIG. 8) engaging the heart wall 200. As shown in FIG. 4C, the incisor 208 is withdrawn and a blood column 214 fills the first conduit 108. The vacuum in the first conduit 108 is adjusted such that a height of the blood column 214 is set at the marker 180. If the incisor 208 is completely removed from the access device 200, a clamp (not shown) is used to seal the elastomer boot 112.

Once the blood column 214 is established, the accessory device, such as an ablation probe 216, is advanced through the column of blood 214 in the first conduit 108 until its distal end 218 is in an interior 212 of the heart 202. The surgeon then manipulates the probe with the help of an imaging means, such as ultrasound or a fluorescent technique to ablate selected tissue within the left ventricle of the heart, for instance to repair an AF defect. Those skilled in the art will appreciate that working against the column of blood eliminates any possibility for air to enter the left ventricle thereby reducing the possibility of creating an embolism.

After completion of the procedure, the accessory device 216 is withdrawn into the first conduit 108 or channel 126 and the purse strings 204 are removed from the suture anchor 156. The vacuum in the interior 166 of the suction cup 162 is shut off or decreased and while maintaining the vacuum in the first conduit 108, the purse strings 204 are pulled together to close the incision 210 simultaneously with the withdrawal of the access device 100. With another device, the incision 210 is completely closed by knotting the purse strings and/or with surgical glue and/or with any other means known to those skilled in the art. The incision 201 in the skin 206 is also closed.

Referring now to FIGS. 5 and 6, there is shown a second preferred implementation of the access device of the present invention, generally referred to by reference numeral 300 and in which adhesive strips 144 are not shown. The access device 300 of FIGS. 5 and 6 is shown with an incisor instrument 208 inserted therein and includes like references to refer to like features of access device 100 previously described. In the access device 300 of FIGS. 5 and 6, a second tubular body 302 is provided for protection of the suction cup 162 during placement of the distal end 106 on the wall of the hollow organ. The second tubular body 302 has a conduit 304 in which the first tubular body 102 is disposed. The second tubular body 302 thus acts as a built-in trocar. The second tubular body 302 is movably disposed on the first tubular body 102 between a first position, shown in FIG. 4B in which the suction cup 162 is disposed within the conduit 304 and a second position in which the suction cup 162 is exposed from the conduit 304 for engagement with the wall of the hollow organ. To facilitate movement of the second tubular body 302 between the first and second positions, a pin 306 is provided on the first tubular body 102. The pin 306 is slidingly engaged with a slot 308 on the second tubular body 302. After the access device 200 has been inserted to contact the wall of the hollow organ, the second tubular body 302 is slid proximally and the pin 306 slides in the same direction along a longitudinal portion 308a of the slot 308 to expose the suction cup 162 from the conduit 304. The second tubular body is then rotated such that the pin 306 slides in a circumferential portion 308b of the slot 308 and is retained there during the remainder of the procedure. The pin 306 may have a threaded knob (not shown) on an end thereof, which when tightened, positively locks the second tubular body 302 in the second position. Furthermore, in the access device 300 of FIGS. 5 and 6, one or more suture slits 157 are disposed on the second tubular body 302 for anchoring the distal end 106 to the wall of the hollow organ with a suture tied between the wall and the suture slit 157. The operation of the access device 300 is otherwise similar to that described above with regard to access device 100.

Referring now to FIGS. 7 and 8, there is shown a third preferred implementation of the access device of the present invention, generally referred to by reference numeral 400, in which like references refer to like features of access device 100 previously described, and in which adhesive strips 144 are not shown. In the access device 400, a second tubular body 402 is movably disposed within a first conduit 404 of a first tubular body 406. The second tubular body 402 has a second conduit 408 for passage of the instrument from the proximal end 104 to the distal end 106 and for maintaining the blood column as previously described. The distal end 106 of the second tubular body 402 includes the extension portion 174 that extends into the opening in the wall of the hollow organ. The proximal end 106 of the second tubular body 402 includes the y-fitting 122 and elastomer boot 112 as previously described with regard to access device 100. The first tubular body 406 includes the suction cup 162 on the distal end 106 as previously described with regard to access device 100. At least one seal 410 is provided in an annular space between the first and second tubular bodies 406, 402 to maintain the integrity of the blood column maintained in the second conduit 408. Seal 410 is preferably an elastomer and also serves to act as a spacer to maintain an equal annular space around a periphery of the first and second tubular bodies 406, 402 and as a linear guide to guide the in and out movement of the second tubular body 402. For this reason, it is preferred that a second seal 410 also be positioned in the proximal end 106 of the access device 400.

The access device 400 further has an extension means for extending the extension portion 174 of the second tubular body 402 into the opening in the wall of the hollow organ. The extension means preferably comprises a nut 412 rotatably retained on the first tubular body 406. The nut 406 having an internal thread 414. The second tubular body 402 having a threaded portion 416 on an exterior surface thereof threadingly engaged with the internal thread 414 of the nut 412. Rotation of the nut 412 causes a relative movement between the first and second tubular bodies 406, 402 such that the extension portion 174 can be extended and retracted relative to the suction cup 162 and first tubular body 406. Preferably, prior to making the incision 210 in the wall 200 of the hollow organ 202, the extension portion is 174 is withdrawn such that it is proximal to a distal-most portion 162a of the suction cup and after providing the vacuum to the interior 166 of the suction cup 162 the extension portion is extended such that it is distal to the distal-most portion 162a of the suction cup. The operation of the access device 400 is otherwise similar to that described above with regard to access device 100.

Several alternatives of the distal end sealing and securing means 154 will now be described with regard to FIGS. 9-16F. Such alternatives employ first and second tubular bodies that are slidingly disposed relative to one another. Only features and functionality which differ from the access devices previously described will be discussed, thus, the access devices described below with regard to FIGS. 9-16F are assumed to have the same features and functionality as described above in addition to the added features and functionality of the alternative sealing and securing means 154. Although such sealing means are preferably used with the access devices described above, they are also useful with access devices in general, including those known in the art.

Referring first to FIG. 9, there is shown an access device 500 having a first alternative distal end sealing and securing means 154. The access device 500 of FIG. 9 is shown as having a different perspective on each end thereof in order to clearly show the features thereof. Access device 500 has a first tubular body 502 movably disposed within a conduit of a second tubular body 504. At a proximal end 104 of the access device 500, each of the first and second tubular bodies 502, 504 have knob portions 506, 508, respectively, for facilitating a relative rotation therebetween. At a distal end 106 of the access device 500, the first and second tubular bodies 502, 504 have flange portions 510, 512, respectively. The first tubular body 502 further has an external thread portion 514 and the second tubular body 504 has a mating internal thread portion (not shown) that threadingly engages with the external thread portion 514.

An operation of the distal end sealing means 154 of the access device 500 of FIG. 9 will now be described with reference to FIGS. 10A-10D. The suture anchor 156 may also be provided as discussed above, but is omitted from the Figures for the sake of clarity. As shown in FIG. 10A, the distal end 106 is contacted to the wall 200 of the hollow organ 202 and an incision is made with the incisor 208 as described above. The incisor is withdrawn as shown in FIG. 10B and the distal end 104 is advanced through the incision as shown in FIG. 10C such that the lip portion 510 of the first tubular body 502 passes through the incision. As shown in FIG. 10D, the first and second tubular bodies 502, 504 are then rotated by a relative rotation of the knob portions 506, 508 to securely and also preferably sealingly clamp the wall 200 between the lip portions 510, 512. Alternatively, the lip portions 510, 512 may only provide a securing means for securing to the wall 200 and a sealing means, such as the suction cup 162, may also be provided to provide the necessary seal. In yet another alternative, the sealing means may be omitted from the access device 500 in cases where embolism control is not necessary.

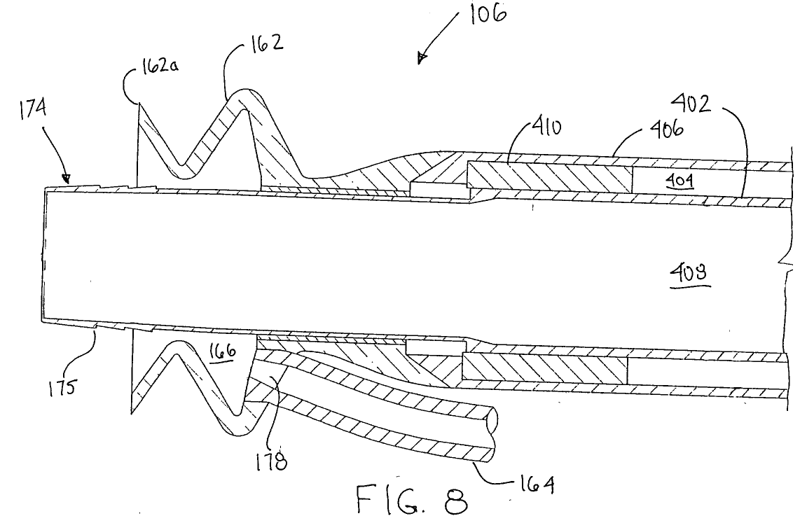

Referring next to FIG. 11, there is shown an access device 600 having a second alternative distal end sealing and securing means 154. The access device 600 of FIG. 11 is also shown as having a different perspective on each end thereof in order to clearly show the features thereof. Access device 600 has a first tubular body 602 movably disposed within a conduit of a second tubular body 604. At a proximal end 104 of the access device 600 each of the first and second tubular bodies 602, 604 have stops 606, 608, respectively, for facilitating a relative translation for containing a compression spring 609 therebetween. At a distal end 106 of the access device 600, the first and second tubular bodies 602, 604 have flange portions 610, 612, respectively. The compression spring 609 biases the stops 606, 608 apart and the flange portions 610, 612 together.

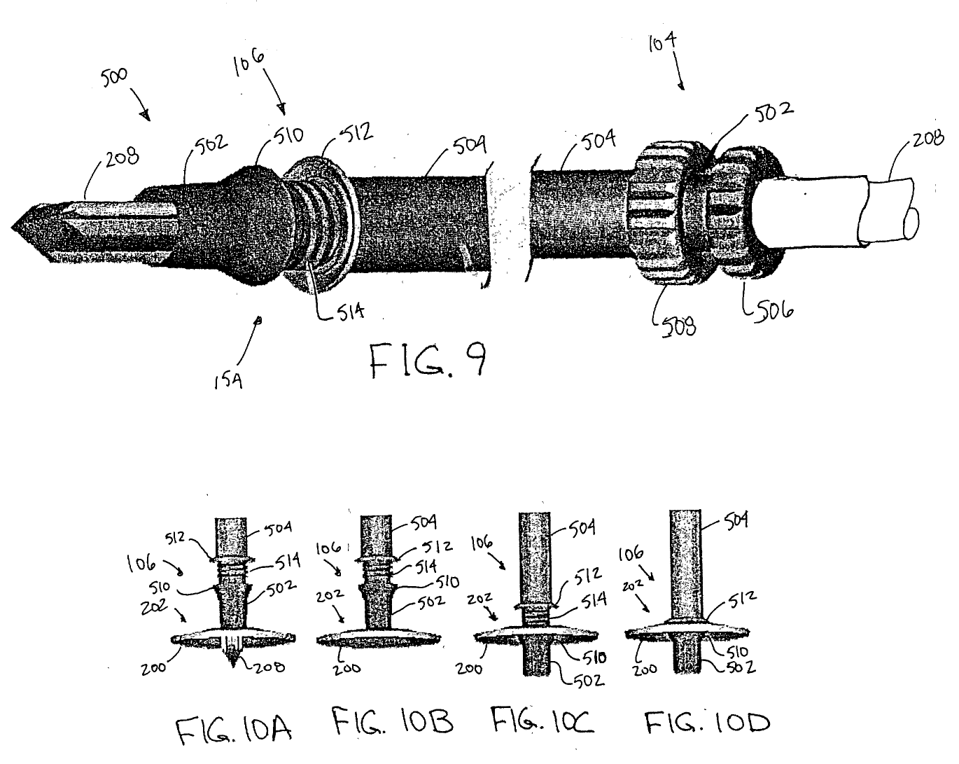

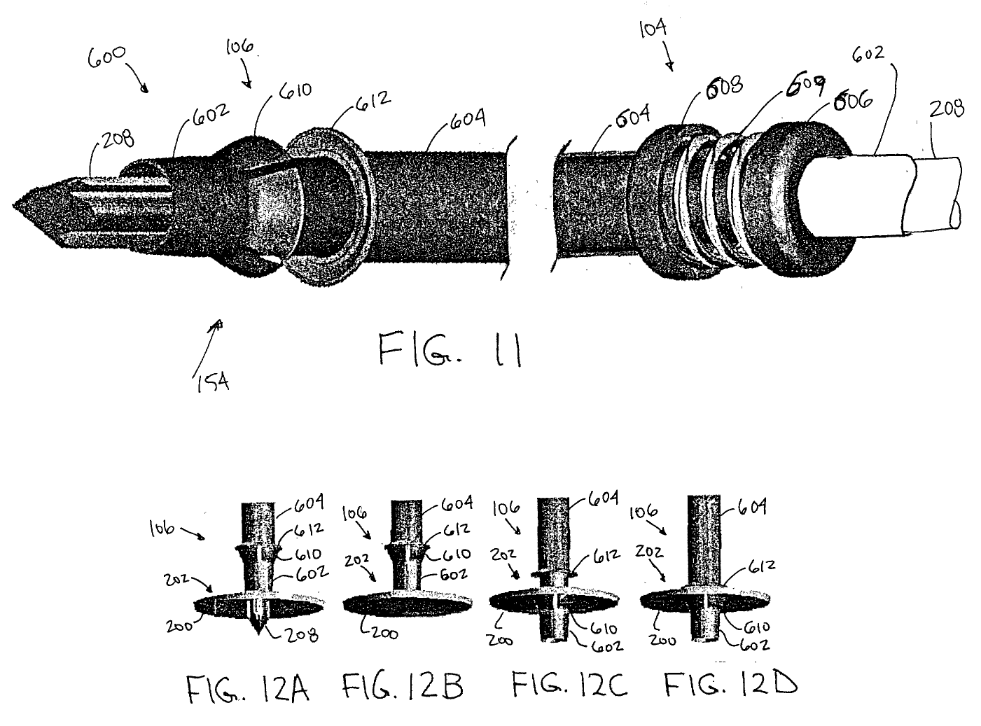

An operation of the distal end sealing means 154 of the access device 600 of FIG. 11 will now be described with reference to FIGS. 12A-12D. The suture anchor 156 may also be provided as discussed above, but is omitted from the Figures for the sake of clarity. As shown in FIG. 12A, the distal end 106 is contacted to the wall 200 of the hollow organ 202 and an incision is made with the incisor 208 as described above. The incisor is withdrawn as shown in FIG. 12B and the stops 606, 608 are urged together against the biasing force of the compression spring 609 to separate the flange portions 610, 612 from each other. The distal end 106 is then advanced through the incision as shown in FIG. 12C such that the lip portion 610 of the first tubular body 602 passes through the incision. As shown in FIG. 12D, the stops 606, 608 are then released and the biasing force of the spring 609 securely and also preferably sealingly clamps the wall 200 between the lip portions 610, 612. Alternatively, the lip portions 610, 612 may only provide a securing means for securing to the wall 200 and a sealing means, such as the suction cup 162, may also be provided to provide the necessary seal. In yet another alternative, the sealing means may be omitted from the access device 600 in cases where embolism control is not necessary.

Referring next to FIGS. 13A-13C, there is shown an access device 700 having a third alternative distal end sealing and securing means 154. The access device 700 of FIGS. 13A-13C has a first tubular body 702 movably disposed within a conduit of a second tubular body 704. At a proximal end 104 of the access device 600 each of the first and second tubular bodies 602, 604 have stops (not shown), respectively, for facilitating a relative translation between the first and second tubular bodies 702, 704. At a distal-most end 706 of the access device 700, the first and second tubular bodies are connected by any means known in the art. The distal end 106 of the second tubular member 704 further has a flange 708 and a flexible portion 710 having a plurality of longitudinal slits 712 spaced around a circumference of the second tubular body 704. The second tubular member 704 may be fabricated entirely from a flexible material or only the flexible portion 710. Furthermore, the second tubular body 704 may be made entirely of an otherwise rigid material that becomes flexible due to the slits 712.

An operation of the distal end sealing means 154 of the access device 700 will now be described with reference to FIGS. 13A-13C. The suture anchor 156 may also be provided as discussed above, but is omitted from the Figures for the sake of clarity. As shown in FIG. 13A, an incision is made in the wall 200 of the hollow organ 202 and the flexible portion 710 of the distal end 106 is then advanced through the incision such that the flange 708 of the second tubular body 704 butts against the wall 200. As shown in FIG. 13B, the incisor 208 is withdrawn and the stops (not shown) or other means are used to facilitate a proximal pulling of the first tubular body 702 relative to the second tubular body 704 to bulge the flexible portion 710 and securely and also preferably sealingly clamps the wall 200 between the bulged flexible portion 710 and the flange 708. Alternatively, the flexible portion 710 and flange 708 may only provide a securing means for securing to the wall 200 and a sealing means, such as the suction cup 162, may also be provided to provide the necessary seal. In yet another alternative, the sealing means may be omitted from the access device 700 in cases where embolism control is not necessary.

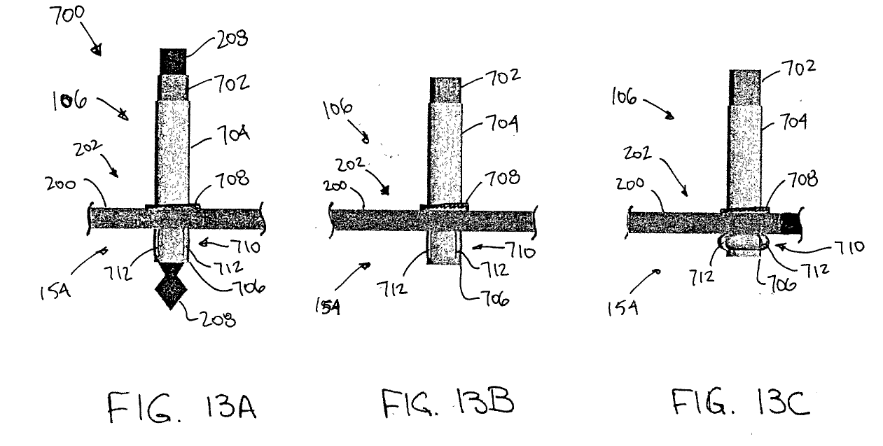

Referring now to FIGS. 14A and 14B there is illustrated a variation of the access device 700, referred to by reference numeral 750. In access device 750, the flexible portion 710 is an elastomer material, such as silicone. The elastomer material is bonded at one end 710a to the second tubular body 704 and at another end 710b to the distal-most end 706 of the first tubular body 702. Similar to access device 700, when the first tubular body 702 is pulled proximally the flexible portion 710 bulges and securely and sealingly clamps the wall 200 between the bulged flexible portion 710 and the flange 708.

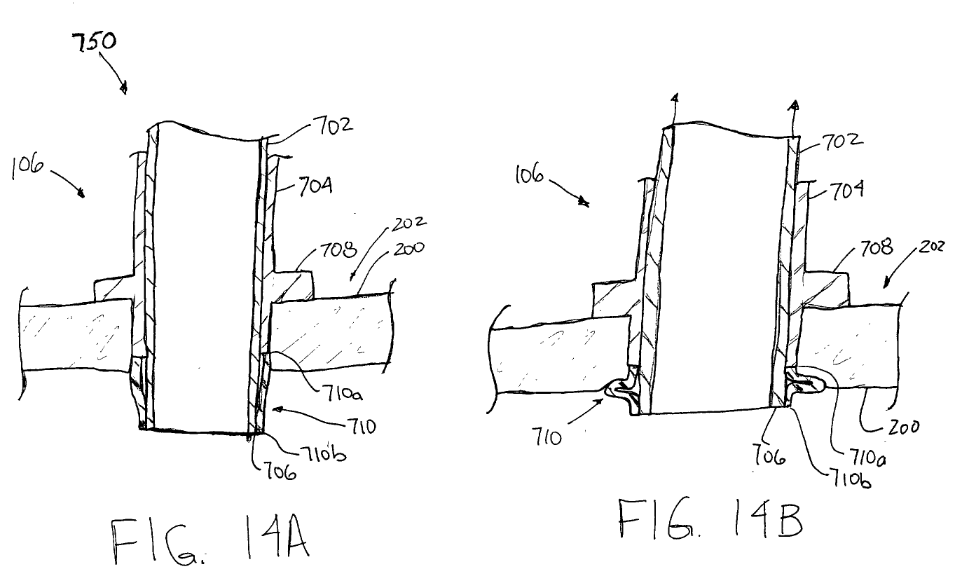

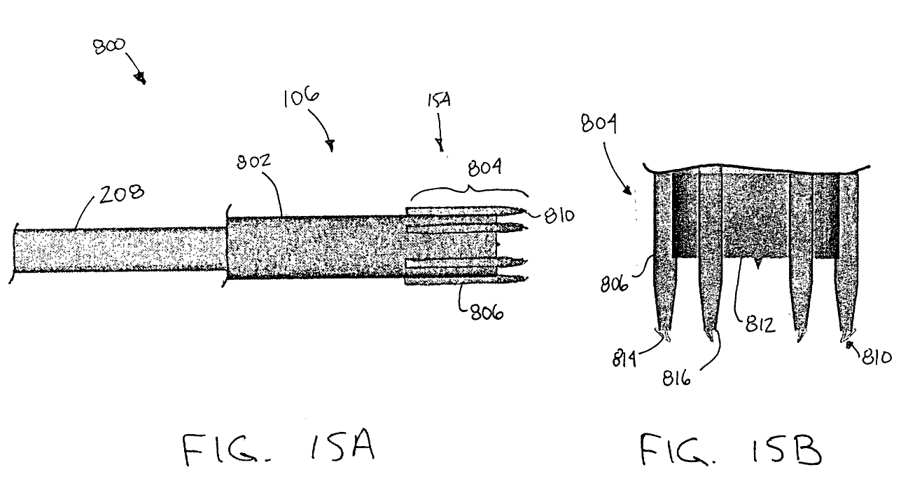

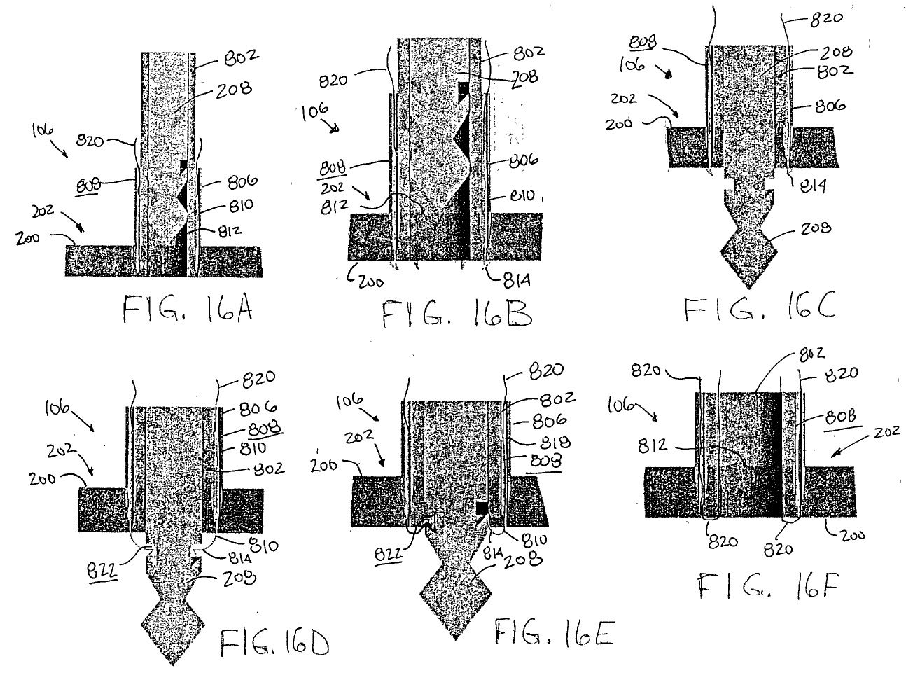

Referring now to FIGS. 15A-16F, yet another distal end sealing and securing means 154 is illustrated therein for use with an access device described above or with access devices of the prior art in general. The access device of FIGS. 15A-16F is referred to generally by reference numeral 800. In the access device 800, a second tubular member is not required because its functions in providing the distal end sealing and securing means may be provided by an instrument, preferably the incisor 208, inserted therein. Therefore, the description of the access device 800 will be made with reference to the incisor 208, however, those skilled in the art will appreciate that any instrument or a second tubular member may also be used in place of the incisor 208.

Referring first to FIGS. 15A and 15B, the access device has a first tubular body 802 having a needle holding section 804 at a distal end 106 thereof. The needle holding section has a plurality of needle holders 806, each having an internal conduit 808 (shown in FIGS. 16A-16F) for housing a needle 810. The needle holders 806 preferably protrude from a distal-most end 812 of the first tubular member 802 and the needles preferably have a hook end 814 that protrudes slightly from a distal-most end 816 of the needle holders 806. The hook ends 814 are normally curved but are retained in the internal conduits 808 in a substantially straight configuration. Upon exiting the conduits 808, the hook ends 814 take their curved shapes (as described below). The needles 810 are preferably fabricated from a super elastic material such as nitinol. Each of the needles 810 have another end 818 which have a suture 820 attached thereto. The suture 820 may be attached by any means known in the art, such as by swaging or by threading and knotting an eyelet. The sutures 820 extend proximally from the conduits 808 and have a proximal free end that is preferably maintained taught.

Referring now to FIGS. 16A-16F an operation of the distal end sealing and securing means 154 of access device 800 will be described. As shown in FIG. 16A, the distal end 106 of the first tubular body 802 is pushed against the wall 200 of the hollow organ 202 such that the portions of the needle holders 806 lodge into the wall 200 and the distal-most end 812 of the first tubular member 802 butts against the wall 200. As shown in FIG. 16B, the hook end 814 that protrudes slightly from the distal-most end 816 of the needle holders 806 are engaged with the interior of the wall 200 by pulling on the sutures to stabilize and secure the distal end 106 of the access device 800 to the wall 200. Once stabilized, the incisor 208 is inserted to make an incision and also preferably to dilate the incision, as shown in FIG. 16C. As shown in FIG. 16D, the needles 810 are inserted distally to expose the hook ends 814 thereof resulting in the hook ends 814 taking their curved shape. Upon the hook ends 814 being released from the conduits 808, a portion of the hook ends 814 is captured by a channeled portion 822 on the incisor 208. As shown in FIG. 16E, the incisor 208 is withdrawn which also pulls the needles 810 from the conduits 808 and creates a sutured butt joint of the distal end 106 of the access device 800 to the wall 200, as shown in FIG. 16F.

Alternatively, the distal end sealing and securing means 154 of access device 800 may only provide a securing means for securing to the wall 200 and a sealing means, such as the suction cup 162, may also be provided to provide the necessary seal. In yet another alternative, the sealing means may be omitted from the access device 800 in cases where embolism control is not necessary.

While there has been shown and described what is considered to be preferred embodiments of the invention, it will, of course, be understood that various modifications and changes in form or detail could readily be made without departing from the spirit of the invention. It is therefore intended that the invention be not limited to the exact forms described and illustrated, but should be constructed to cover all modifications that may fall within the scope of the appended claims.

What is claimed is:

1. An access device for providing endoscopic access into an interior of a hollow organ of a patient, the access device comprising:
a first tubular body, the first tubular body extending from a proximal end at an exterior surface of the patient to a distal end at the hollow organ, the first tubular body further having a first conduit for passage of an instrument from the proximal to the distal end;
distal end securing means for at least one of sealing and securing the distal end of the first tubular body to a wall of the hollow organ; and
proximal end securing means for securing the proximal end of the first tubular body to a skin of the patient.

2. The access device of claim 1, wherein the first tubular body further comprises a proximal seal on the proximal end for sealing against an instrument passed through the first conduit.

3. The access device of claim 2, wherein the proximal seal is an elastomer boot having a second conduit in fluid communication with the first conduit and an opening to the second conduit having a size substantially equivalent to an outer size of the instrument.

4. The access device of claim 1, further comprising at least one access port in communication with the first conduit.

5. The access device of claim 4, wherein the at least one access port comprises a y-fitting disposed at the proximal end of the first tubular body, the y-fitting having first and second access ports, each of which having a channel in communication with the first conduit.

6. The access device of claim 5, wherein at least one of the first and second access ports further comprise an accessory seal disposed in or on the corresponding channel for sealing against an accessory instrument inserted therein.

7. The access device of claim 6, wherein the accessory seal comprises a sealing cap having a bore with at least a portion of the bore substantially corresponding to an outer size of the accessory instrument, the sealing cap further having a slot extending from an exterior surface of the sealing cap to the bore for facilitating insertion of the accessory instrument into the bore.

8. The access device of claim 1, wherein the distal end securing means at least secures the distal end to the wall of the hollow organ and wherein the distal end securing means comprises a suture anchor disposed at the distal end of the first tubular body for anchoring the distal end to the wall of the hollow organ with at least one suture engaging the wall and the suture anchor.

9. The access device of claim 8, wherein the suture anchor has at least one of slits and holes for engaging with the at least one suture.

10. The access device of claim 1, wherein the distal end securing means at least seals the distal end to the wall of the hollow organ.

11. The access device of claim 10, wherein the distal end securing means comprises:
a suction cup disposed at the distal end of the first tubular body for engaging with the wall of the hollow organ; and
a conduit in fluid communication with an interior of the suction cup for providing a vacuum in the interior of the suction cup to thereby secure and seal the distal end of the first tubular body to the wall of the hollow organ.

12. The access device of claim 1, wherein the proximal end securing means comprises at least one adhesive strip having a free end and a fixed end fixed to the proximal end of the first tubular body, the at least one adhesive strip having an adhesive on at least one surface thereof for attaching to the skin of the patient.

13. The access device of claim 1, wherein the hollow organ is the heart and the access device further comprises embolism control means for reducing the possibility of introducing an embolism into the heart.

14. The access device of claim 13, wherein the distal end securing means at least seals the distal end to the wall of the heart and the embolism control means comprises a vacuum port in communication with the first conduit for applying a vacuum to the first conduit to draw and maintain a column of blood from the heart into at least a portion of the first conduit.

15. The access device of claim 14, wherein at least a portion of the first tubular body comprises a transparent material for visualizing a height of the column of blood.

16. The access device of claim 15, further comprising a marker disposed on the transparent material for indicating a desired height of the column of blood.

17. The access device of claim 14, wherein the embolism control means further comprises an anticoagulation coating disposed on at least an interior surface of the first conduit.

18. The access device of claim 11, further comprising a second tubular body for protection of the suction cup during placement of the distal end on the wall of the hollow organ, the second tubular body having a second conduit in which the first tubular body is disposed, the second tubular body being movably disposed between a first position in which the suction cup is disposed within the second conduit and a second position in which the suction cup is exposed for engagement with the wall of the hollow organ.

19. The access device of claim 18, wherein the distal end securing means at least secures the distal end to the wall of the hollow organ and wherein the distal end securing means comprises a suture anchor disposed on the second tubular body for anchoring the distal end to the wall of the hollow organ with at least one suture engaged with the wall and the suture anchor.

20. The access device of claim 19, wherein the suture anchor has at least one of slits and holes for engaging with the at least one suture.

21. The access device of claim 1, wherein the distal end of the first tubular body includes an extension portion that extends into the opening in the wall of the hollow organ.

22. The access device of claim 21, wherein the extension portion comprises one or more barbed edges on an exterior of the extension portion of the first tubular body for engaging with the opening in the wall of the hollow organ.

23. The access device of claim 21, wherein the distal end securing means at least seals the distal end to the wall of the hollow organ and wherein the distal end securing means comprises;
a suction cup disposed at the distal end of the first tubular body for engaging with the wall of the hollow organ; and
a conduit in fluid communication with an interior of the suction cup for providing a vacuum to the interior of the suction cup to thereby secure and seal the distal end of the first tubular body to the wall of the hollow organ;
wherein the extension portion is distal to the suction cup.

24. The access device of claim 1, further comprising a second tubular body movably disposed within the first conduit, the second tubular body having a second conduit for passage of the instrument from the proximal end to the distal end, wherein a distal end of the second tubular body includes an extension portion that extends into the opening in the wall of the hollow organ.

25. The access device of claim 24, wherein the extension portion comprises one or more barbed edges on an exterior of the extension portion of the second tubular body for engaging with the opening in the wall of the hollow organ.

26. The access device of claim 24, further comprising extension means for extending the extension portion of the second tubular body into the opening in the wall of the hollow organ.

27. The access device of claim 26, wherein the extension means comprises:
a nut rotatably retained on the first tubular body, the nut having an internal thread; and
the second tubular body having a threaded portion on an exterior surface thereof threadingly engaged with the internal thread of the nut;
wherein rotation of the nut causes a relative movement between the first and second tubular bodies.

28. The access device of claim 24, wherein the distal end securing means at least seals the distal end to the wall of the hollow organ and wherein the distal end securing means comprises:
a suction cup disposed at the distal end of the second tubular body for engaging with the wall of the hollow organ; and
a conduit in fluid communication with an interior of the suction cup for providing a vacuum to the interior of the suction cup to thereby secure and seal the distal end of the second tubular body to the wall of the hollow organ;
wherein the extension portion is distal to the suction cup when in an extended position.

29. The access device of claim 1, wherein the instrument is an incisor having a cutting blade and disposed in the first conduit such that the cutting blade extends from the distal end of the first tubular body to contact the wall of the hollow organ and to incise the opening therein.

30. The access device of claim 29, wherein the incisor further has a dilator for dilating the opening after incision.

31. An access device for providing endoscopic access into an interior of a heart of a patient, the access device comprising:
a first tubular body, the first tubular body extending from a proximal end at an exterior surface of the patient to a distal end at the heart, the first tubular body further having a first conduit for passage of an instrument from the proximal to the distal end;
distal end securing means for at least one of sealing and securing the distal end of the first tubular body to a wall of the heart; and
embolism control means for reducing the possibility of introducing an embolism into the heart.

32. The access device of claim 31, wherein the distal end securing means at least seals the distal end to the wall of the heart and the embolism control means comprises a vacuum port in communication with the first conduit for applying a vacuum to the first conduit to draw a column of blood from the heart into at least a portion of the first conduit.

33. The access device of claim 32, wherein at least a portion of the first tubular body comprises a transparent material for visualizing a height of the column of blood.

34. The access device of claim 33, further comprising a marker disposed on the transparent material for indicating a desired height of the column of blood.

35. The access device of claim 32, wherein the embolism control means further comprises an anticoagulation coating disposed on at least an interior surface of the first conduit.

36. An access device for providing endoscopic access into an interior of a hollow organ of a patient, the access device comprising:
a first tubular body, the first tubular body extending from a proximal end at an exterior surface of the patient to a distal end at the hollow organ, the first tubular body further having a first conduit for passage of an instrument from the proximal to the distal end;
a suction cup disposed at the distal end of the first tubular body for engaging with the wall of the hollow organ; and
a conduit in fluid communication with an interior of the suction cup for providing a vacuum in the interior of the suction cup to thereby secure and seal the distal end of the first tubular body to the wall of the hollow organ.

37. A method for providing endoscopic access into an interior of a hollow organ of a patient, the method comprising:
at least one of sealing and securing a distal end of an access device to a wall of the hollow organ; and
securing a proximal end of the access device to a skin of the patient.

38. A method for providing endoscopic access into an interior of a heart of a patient, the method comprising:
sealing a distal end of an access device to a wall of the heart; and
maintaining a column of blood from the heart into at least a portion of an access device to reduce the possibility of introducing an embolism into the heart.

39. The method of claim 38, wherein the maintaining comprises applying a vacuum to an instrument conduit in the access device.

40. The method of claim 39, further comprising controlling an amount of vacuum to vary a height of the column of blood in the instrument conduit.

41. A method for providing endoscopic access into an interior of a hollow organ of a patient, the method comprising:
disposing a distal end of an access device on a wall of the hollow organ; and
vacuum sealing the distal end to the wall with a suction cup disposed at the distal end of the access device to thereby secure and seal the distal end to the wall of the hollow organ.
